(19) United States
(12) Patent Application Publication
(10) Pub. No.: US 2003/0060397 A1
(43) Pub. Date: Mar. 27, 2003

(54) Method of treating and preventing acute neural lesions with substances that modulate the expression or function of a protein involved in the cell cycle and pharmaceutical preparations containing such substances

(75) Inventors: Serge Timsit, Paris (FR); Pauline Cavelier, Strasbourg (FR); Yehezkel Ben-Ari, Marseille (FR); Michel Khrestchatisky, Auriol (FR); Laurent Meijer, Roscoff (FR)

Correspondence Address: SCHNADER HARRISON SEGAL & LEWIS, LLP, 1600 MARKET STREET, SUITE 3600, PHILADELPHIA, PA, 19103

(73) Assignee: Centre National de la Recherche Scientifique - CNRS and Inserm, Paris Cedex (FR)

(21) Appl. No.: 10/251,297

(22) Filed: Sep. 20, 2002

Related U.S. Application Data
(63) Continuation of application No. PCT/FR01/00850, filed on Mar. 21, 2001.

(30) Foreign Application Priority Data
Mar. 22, 2000 (FR) 00/03673

(51) Int. Cl. A61K 31/00

(52) U.S. Cl. 514/1

(57) ABSTRACT

A method of treating or preventing non-apoptotic excitotoxic acute neural lesions in a patient including administering a therapeutically effective amount of a substance that modulates expression or the function of a protein involved in cell cycles, and a pharmaceutical preparation for treating or preventing non-apoptotic excitotoxic acute neural lesions including a therapeutically effective amount of a substance that modulates expression or the function of a protein involved in the cell cycle.

### RELATED APPLICATION

This is a continuation of International Application No. PCT/FR01/00850, with an international filing date of Mar. 21, 2001, which is based on French Patent Application No. 00/03673, filed Mar. 22, 2000.

### FIELD OF THE INVENTION

This invention relates to the treatment and prevention of neurodegenerative diseases linked to excitotoxic acute neural lesions. The invention pertains more specifically to the treatment and prevention of epilepsy, and more specifically to status epilepticus. The invention also especially pertains to the treatment and prevention of cerebral ischemia, whether it be focal or global cerebral ischemia, cerebral hypoxia subsequent to cardiac arrest, extracorporeal circulation during cardiovascular surgery, surgery of the vessels of the neck, possibly requiring clamping of the vessels, cranial trauma and any situation causing cerebral hypoxia or anoxia.

### BACKGROUND

Destruction of cerebral tissue can occur over the course of various morphologic phenomena. Apoptosis is a mechanism of cell death which developed with the birth of multicellular organisms. In this initial description, apoptosis is a physiological phenomenon which is found through the entire phylogeny. In this context, the structure of the brain is a striking example. The brain can form a structure during development as a result of the massive death of neurons (more than 50%).

The term apoptosis is derived from the Greek for “falling leaves” described by Kerr (1972). It refers to the different morphologic criteria of necrosis. In electronic microscopy, apoptosis is characterized at an early stage by condensation of the cytoplasm and chromatin and, later, by the occurrence of convolutions of the cytoplasmic and nuclear membranes which then form apoptotic bodies. Physiologically, apoptosis does not cause inflammation. It was found that apoptosis was associated generally, but not necessarily, with characteristic biochemical phenomena bringing into play a veritable program of death referred to in a consecrated manner as programmed cell death (PCD). The term “PCD” has two meanings. The first historically refers to a death predicted over the course of the development. The term was subsequently modified to indicate that it is associated with a genetic program involving the synthesis of specific proteins.

Necrosis, in turn, is characterized by swelling of intracellular organelles and cytoplasm, and then an osmotic lysis. Liberation of these constituents causes an afflux of macrophages and tissue lesions. Thus, inflammation is present at the heart of the necrosis which is most often a pathological phenomenon.

Thus, death from necrosis and death from apoptosis are associated classically with passive and active phenomena, respectively. The active phenomena bring into play a cell death program with activation of proteins (caspase family, Bcl-2 family), whereas the passive phenomena do not bring into play a cell death program.

Thus, there is on the one hand the morphologic aspects and, on the other, the biological phenomena playing a role in cell death. It has been thought for a long time that the morphologic aspects involved specific biological mechanisms. However, this belief is presently being modified. The programmed apoptosis-death, programmed necrosis-absence of death concept is no longer viable. For example, caspase-dependent apoptoses have been reported as have caspase-independent apoptoses as well (Borner et al., 1999). There are forms of passages between apoptosis and necrosis, as well as cells in apoptosis for which the programmed death was blocked and which can have the morphologic characteristics of necrosis (Kitanaka et al., 1999; Chautan et al., 1999).

Over the course of cerebral ischemia, the morphologic appearances are sometimes suggestive of apoptosis and sometimes of necrosis, independent of the fact of whether or not programmed death is present. It is not even certain that there are neurons which die from classic apoptosis over the course of cerebral ischemia (MacManus et al., 1999). Research carried out by Portera-Cailliau et al. (1997) illustrates the morphologic continuum that can exist after excitotoxicity between necrosis and apoptosis. These authors injected into the striatum various glutamatergic agonists to stimulate the NMDA and non-NMDA receptors. They then studied the morphologic appearance of the neurons. After excitotoxic lesion, the intermediary appearances between necrosis and apoptosis could be seen. After injection of NMDA, the cell morphology was rather of necrotic type, whereas after injection of non-NMDA agonists, it was rather of apoptotic type.

The invention is based on the comprehension of molecular mechanisms involved in neuronal death and, in particular, the neural death linked to the phenomenon of excitotoxicity. Neuronal death linked to excitotoxicity is due to an excessive liberation of glutamate which then leads to lesions. The death associated with excitotoxicity can cause a programmed type death that can bring into play gene product activation. This programmed type death can be associated from a morphologic point of view over the course of the excitotoxicity and the cerebral ischemia with various morphologic appearances of necrosis, apoptosis and autophagocytosis as well as mixed aspects (apoptosis/necrosis). This phenomenon is found over the course of ischemia and epilepsy and in numerous neurodegenerative diseases such as Parkinson's disease, Huntington's disease and amyotrophic lateral sclerosis.

Other cells of the central nervous system can also be sensitive to excitotoxicity. For example, oligodendrocytes subjected to glutamatergic agonists such as kainate can also degenerate (Matute et al., 1997; Sanchez-Gomez and Matute, 1999).

The inventors focused most specifically on the acute neural lesions characteristic of epilepsy and cerebral ischemia, whereas the neurodegenerative diseases of the Alzheimer's or Parkinson's type are chronic diseases with an essentially progressive neuronal death over many years.

In the case of epilepsy and cerebral ischemia, the neural death is acute and two types of neural lesions are observed:

the death of neurons, astrocytes and oligodendrocytes;

the proliferation of inflammation cells, in particular, astrocytes and microglia, which by their inflammatory effects have a deleterious effect on cell death (Zoppo et al., 2000). Cells outside of the central nervous system can also come into question, such as endothelial cells and leukocytes.

It is known that cyclins are key molecules in the cell cycle, involved in the phosphorylation of the Rb molecule to enable progression of the cell cycle. Their mitotic properties require them to be associated with CDKs (cyclin dependent kinases) to form the complexes responsible for the phosphorylation of the Rb molecules. The D cyclins can also acts independently of CDKs as has been demonstrated in recent studies (Zwijsen et al, 1997).

In fact, the CDK inhibitors are known for their antimitotic property and have already been proposed as anticancer agents and for preventing and treating tissue degeneration, especially apoptosis of neuronal cells. Thus, multiple PCT international patent applications (WO 99/43676 and WO 99/43675) have proposed CDK inhibitors as inhibitors of the progression of the cell cycle for use in the treatment and prevention of neuronal apoptosis, e.g., for cerebrovascular diseases.

Also previously proposed was the use of the inhibitor of GSK3 for protecting neurons (Maggirwar, S. B. et al., 1999, J. Neurochem. 73, 578-586).

The role of cyclins in cerebral ischemia and excitotoxicity remains controversial. Certain authors believe that cyclin D1 is associated with neuronal repair, whereas others believe that it could be involved in neuronal death. Wiessner et al. (1996) detected in vivo the presence of cyclin D1 in the microglia, but not in the neurons after global cerebral ischemia. Li et al. (1997) observed that the protein cyclin D1 was augmented in the neurons and oligodendrocytes after focal ischemia. Since these cells were not in a state of degeneration, the authors proposed that cyclin D1 could be involved in the repair of DNA in the neurons not attacked in an irreparable manner. Small et al. (1999) studied in vitro the expression of cyclin D1 on a culture of cortical neurons exposed to glutamate. They observed a loss of expression of cyclin D1 after exposure of these neurons to glutamate and concluded that cyclin D1 plays a role in the neuronal resistance to ischemia.

In a model of global ischemia, Timsit et al. (1999) demonstrated that the expression of mRNA and the protein cyclin D1 was augmented in the neurons destined to die, but also in the resistant neurons. They then proposed that cyclin D1 could be a modulator of programmed death, but could not determine a deleterious or beneficial effect. Recent in vitro results suggest that cyclin D1 and its partners could have a deleterious effect on neuronal death.

Timsit et al. had thus demonstrated an augmentation of the expression of cyclins, more specifically of cyclin D1, in neurons in the context of ischemia or epilepsy (Timsit, S. et al., 1999, Eur. J. Neurosci. 11: 263-278). This in vivo observation was confirmed on an in vitro model of neuronal death. Nevertheless, this finding still appears to contradict many articles in which it is claimed that cyclin D1 is not involved in apoptosis.

### SUMMARY OF THE INVENTION

This invention relates to a method of treating or preventing non-apoptotic excitotoxic acute neural lesions in a patient including administering a therapeutically effective amount of a substance that modulates expression or the function of a protein involved in cell cycles.

This invention also relates to a pharmaceutical preparation for treating or preventing non-apoptotic excitotoxic acute neural lesions including a therapeutically effective amount of a substance that modulates expression or the function of a protein involved in the cell cycle.

### BRIEF DESCRIPTION OF THE DRAWINGS

FIG. 1 is a graph of exposure time to kainate versus the percent of neurons in the state of degeneration.

FIG. 2 shows photographs of phase contrast microscope observations at 22 hours (FIGS. 2A-F) and 5 hours (FIGS. 2G-I) after exposure to 20 μM of kainate and cultures not exposed to kainate (FIGS. 2J-L).

FIG. 3A is a graph of exposure time to kainate versus percent of the control.

FIG. 3B is a Western-blot analysis from protein extracts from hippocampus cells exposed to kainate using an anti-cyclin D1 monoclonal antibody and a Class III anti-β-tubulin antibody.

FIG. 4 is a series of microphotographs showing the expression of CDK5 in neurons after exposure to kainate.

FIG. 5 is a graph of concentration of a roscovitine analog versus percent of neurons in a state of degeneration.

### DETAILED DESCRIPTION

The inventors have now demonstrated on the model defined above that the use of CDK-inhibiting substances leads to a decrease in excitotoxic acute neuronal death.

In the case of the chronic lesions found, e.g., in Parkinson's or Alzheimer's disease, the drug comprising a CDK-inhibiting substance is administered on a chronic basis with side effects on cell division. In contrast, in the case of the acute lesions found in cerebral ischemia and epilepsy, a drug is administered for a short period of time and thus with a low level of side effects on cell division.

Thus, an aspect of the invention relates to a substance that modulates the expression or the function of a protein involved in the cell cycle in a pharmaceutical composition intended for use in the treatment or prevention of non-apoptotic excitotoxic acute neural lesions.

The term “neural lesions” is understood to mean the lesions that can destroy all of the types of cells of the nervous system and, more particularly, the neurons, astrocytes, oligodendrocytes and the microglia, as well as their precursors in the nervous system, including the stem cells which could give rise to astrocytes, oligodendrocytes, neurons and microglia.

These lesions are those found specifically in ischemia or epileptic seizures. They are caused, at least in part, by the phenomenon of excitotoxicity. Thus, they refer to pathological phenomena that are well known in human pathology and not to morphologic appearances. The morphologic appearances can be close to the appearance of necrosis, apoptosis, mixed necrosis/apoptosis appearance and death by autophagocytosis. Among the lesions of necrosis, pale cell change, ischemic cell change and ghost cells have been described. Finally, recent reviews suggest the possibility of passage between different forms of death: necrosis and apoptosis (Lipton et al., Physiological Review 1999; 79: 1432-1532).

The term “acute lesions” is understood to mean all lesions which are produced in less than about 30 days which are in this context due to cerebral ischemia or epileptic seizures.

Thus, the invention pertains more particularly to a substance that modulates the expression or the function of a protein involved in the cell cycle in a pharmaceutical composition intended for use in the treatment or prevention of non-apoptotic excitotoxic acute neural lesions of the neurons, astrocytes or oligodendrocytes, or their precursors over the course of cerebral ischemia or epilepsy, more particularly, status epilepticus.

The invention pertains most specifically to the treatment or prevention of non-apoptotic excitotoxic acute neural lesions occurring over the course of a situation causing cerebral hypoxia or anoxia. The situations causing cerebral hypoxia or anoxia include cardiac arrest, the implementation of extracorporeal circulation during cardiovascular surgery, surgery of the vessels of the neck possibly requiring clamping of the vessels and cranial trauma.

The term “protein involved in the cell cycle” is understood to mean any protein that plays a role in the cell cycle on certain cell types. “Cell cycle” is understood to mean phase G1, phase S, phase G2 and phase M as well as phase G0. Thus, “protein involved in the cell cycle” is understood more particularly to mean a protein that plays a role in the progression of the cell cycle, i.e., passage from one phase to another. This is a protein that can be produced by a type of cell that no longer divides. For example, a differentiated neuron does not divide although it can express certain molecules of the cell cycle without, however, these molecules causing cell division. Moreover, a protein involved in the progression of the cell cycle of one type of cell can be produced by another type of cell.

The term “substance that modulate the expression” is understood to mean any substance capable of modifying the quantity of mRNA produced, the quantity of protein produced or of modifying the half-life of a mRNA or of a protein, e.g., by modifying the degradation of the mRNA or the degradation of the protein. This modulation can be positive or negative, i.e., it can augment or diminish the quantity of active protein.

The term “substance that modulates the function of a protein” is understood to mean any substance capable of modifying the activity of a protein or of a protein complex on a target. The invention pertains more particularly to a substance capable of modulating the phosphorylation of a target by augmenting or inhibiting the phosphorylation. As a preferred example, the invention concerns a substance capable of modulating the degree of phosphorylation of Rb by a CDK.

The invention concerns more particularly the use of a substance that modulates the expression or function of a cyclin, and more specifically of a D cyclin, a CDK or their complex. The term “substance that modulates the expression or function of a cyclin, a CDK or their complex” also means any substance that modulates the expression or function of any complex involving the cyclin, the CDK or both. Examples of complexes are: cyclin/another protein or complex of proteins; CDK/another protein or complex of proteins; cyclin/CDK/another protein or complex of proteins.

The invention pertains most specifically to a substance that modulates the expression or function of cyclin D1 and/or CDK5 and/or the cyclin D1 /CDK5 complex.

These substances exhibit an effect on neuronal excitotoxicity and, thus, on neuronal death, but also on the death of astrocytes and oligodendrocytes and on the indirect deleterious effect linked to the proliferation of astrocytes and microglia involved in the excitotoxicity phenomenon.

The invention thus pertains most particularly to the treatment or prevention of cerebral ischemia and epilepsy. In fact, this invention demonstrates that a substance that modulates the expression or the function of cyclins, CDKs or their complex enables diminishment of the extent of the lesions caused by ischemia or epilepsy. The target cells which are targeted in accordance with aspects of the invention are, on the one hand, the neurons and possibly other cells that die such as astrocytes, oligodendrocytes and microglia and, on the other hand, the cells that proliferate and which have a deleterious effect on the extent of the lesions. The invention thus pertains to a method for treating or preventing cerebral ischemia or epilepsy comprising administration to a patient of a quantity that is effective on the acute neural lesions of one or more of the substances that modulate the expression or function of a protein involved in the cell cycle.

As a substance that modulates the expression or the function of a protein involved in the cell cycle, the invention envisages:

the inhibitors of the expression of cyclins,

the inhibitors of cyclin dependent kinases such as, e.g., the analogues of purines, the derivatives of olomoucine and roscovitine, the paullones, the indirubins, hymenisaldisine, flavopiridol and the like,

the inhibitors of the cyclin/cyclin dependent kinase complex.

The inhibitors of the expression of cyclins are, e.g.:

Rapamycin which acts on the mRNA of cyclin D1 and on the stability of the protein (Hashemolhosseini et al., 1998). It has also been demonstrated that rapamycin can diminish the size of cerebral infarctions.

Glycogen synthase kinase, such as GSK3, which regulates the proteolysis of cyclin D1 (Diehl et al., 1998).

The statins and, in particular, lovastatin which modifies the expression of cyclin D1 (Oda et al., 1999, Rao et al., 1999; Muller et al., 1999) via inhibitory proteins such as p21.

Other advantages and characteristics of the invention will become apparent from the description below concerning the effect of kainate on neuronal death and the role of CDK inhibitors on this neuronal death.

I—Materials and Methods

1) Primary Hippocampus Cell Cultures

Cell cultures were prepared from Wistar rats aged 2 days. The hippocampus was dissected in PBS without calcium or magnesium. The tissues were cut up into small pieces and incubated in the presence of proteases and DNAse. The action of the proteases was stopped by the action of a serum. The cells were dissociated mechanically, then resuspended in a culture medium. Cells cultured for 10-12 days were used for the experiments.

2) Exposure to Kainate and Study of the Cell Mortality

The hippocampus cells were exposed to kainate (20-75 μM) for various periods of time (2-22 hours). The kainate was diluted in water to prepare a stock solution of 20 mM. The required amount of stock solution was then added to 200 μl of processed medium originating from the cell culture. The control experiments were conducted under the same conditions except that the kainate was replaced by sterile water.

Neuronal death was analyzed by phase contrast microscopy and the use of two death markers: propidium iodine and Hoechst dye (Bisbenzimide).

Counting was performed on hippocampus cultures exposed to 20 μM of kainate. At least two dishes per condition were evaluated.

Propidium iodine (7.5 μM) was added to the culture 1 hour prior to cell counting. The labeled cells were counted with a fluorescence microscope with a slight enlargement from fields selected at random. At least 5 fields in two dishes were counted per condition on three independent cultures. The results were expressed as the percentage of the total number of neurons observed with phase contrast microscopy.

Hoechst labeling (Bisbenzimide) was performed after fixation of the cells with 4% paraformaldehyde. The brilliant cells with condensed nuclei were then counted. At least 5 fields in two dishes were counted per condition on one or three independent cultures. The results were expressed as the percentage of the total number of neurons observed with phase contrast microscopy.

3) Immunocytochemistry and Hoechst Dye (Double Labeling)

Hippocampus cells on glass slides were fixed in 4% paraformaldehyde for 20 minutes then washed in PBS and permeabilized in 0.2% PBS—0.2% gelatin Triton X-100. A monoclonal antibody directed against cyclin D1 (Santa Cruz, Calif., USA) diluted to 1/400 and a polyclonal rabbit antibody (Dako A/S, Denmark) directed against GFAP diluted to 1/800 were incubated overnight at 4° C. in 0.2% PBS—0.2% gelatin Triton X-100. After washing, an equine anti-mouse antibody diluted to 1/400 (adsorbed in a rat) (Vector, Burlingame, USA) was used for 1 hour at ambient temperature. After washing, an avidin-fluorescein complex (1/400) was used at the same time as a goat anti-rabbit antibody coupled to rhodamine (Chemicon, Temecula, USA) for an incubation of 1 hour. After washing, the cells were dyed with the Hoechst Bisbenzimide 33.258 (Sigma, St. Louis, USA) at 1 mg/ml. The glass slides were then mounted. The control experiments were performed while omitting the first antibodies, either the cyclin D1 or the GFAP or both.

For the cyclin D1/CDK5 double labeling, an anti-cyclin D1 monoclonal antibody diluted to 1/100 (Santa Cruz, Calif., USA) as well as an anti-CDK5 rabbit polyclonal antibody diluted to 1/200 was used. After washing, an anti-rabbit biotinylated antibody diluted to 1/400 (Vector, Burlingame, USA) was used for 1 hour at ambient temperature. After washing, an anti-mouse goat antibody coupled to TRITC (1/400) (Sigma, St. Louis, USA) and an avidin-fluorescein complex (1/400) (Vector, Burlingame, USA) was used at ambient temperature for 30 minutes. The control experiments were performed by omitting the first antibody, either the cyclin D1 or the CDK5, or both. Another type of control was performed by neutralizing the anti-CDK5 antibody with a 10-fold excess (weight/weight) of immunizing peptide for 30 minutes at 30° C.

4) Western Blot

After exposure of the hippocampus cells to kainate, the cells were washed in PBS then lysed in a Laemli buffer. The samples were subjected to sonication and heated at 100° C. for 5 minutes. Electrophoresis with a 12% SDS-polyacrylamide gel was then performed. The proteins were then transferred onto a nitrocellulose membrane and incubated with an anti-cyclin D1 monoclonal antibody or with an anti-CDK5 polyclonal antibody (Santa Cruz, Calif., USA) or with a class III anti-β-tubulin monoclonal antibody (Sigma, St. Louis, USA), a specific neuronal marker. Labeling was performed using the anti-rabbit antibody or anti-mouse antibody coupled to horseradish peroxidase using the ECLTM kit (Amersham Corp., England). The control experiments were performed by omitting the first antibodies.

5) Immunoprecipitations and Western-Blot Analysis

Rat brains were ground in an RIPAE buffer (PBS containing 1% Triton X-100, 0.1% SDS, 5 mM EDTA, 1% aprotinin and 1% sodium deoxycholate). The clarified lysates were then incubated for 2 hours in the freezer with an anti-CDK5 antibody in the presence or absence of the corresponding blocking peptide. The resultant immune complexes were then recovered by precipitation with Sepharose A protein (Pharmacia) and washed 3 times with RIPAE buffer. The immunoprecipitated proteins were then eluted by boiling them in Laemmli buffer, fractionated over an SDS-polyacrylamide gel and transferred onto a membrane (Immobilon-P, Millipore Corp.). The membranes were then saturated with a blocking solution (5% skimmed milk in 20 mM Tris-HCl, pH 7.6, 0.9% NaCl, 0.2% Tween-20) and incubated with either anti-cyclin D1 (1/200) or anti-CDK5 (1/200) overnight at 4%. Immunolabeling was performed with the antibodies coupled to horseradish peroxidase using the ECLTM kit (Amersham Corp.).

6) Treatment with CDK Inhibitor (ML-1437)

The hippocampus cultures were exposed to kainate (20 μM) in DMSO for 5 hours in the presence or absence of a CDK inhibitor, an analogue of roscovitine. The CDK inhibitor was used at various concentrations: 2 μM, 5 μM and 10 μM. Cell mortality was determined using propidium iodine as described above.

II—Results

1) Neuronal Death After Exposure to Kainate was Delayed and Dose Dependent

Two approaches were used to evaluate neuronal death after exposure to kainate:

morphologic analysis;

the use of dead cell markers: propidium iodine and the Hoechst dye.

i) Morphologic Analysis

Quantitative morphologic analysis was performed on the surviving neurons at different time periods between 1 and 27 hours.

Neuron counting after exposure to 20 μM revealed a very strong drop in neuronal viability between 1 and 5 hours.

ii) Markers of Cell Death

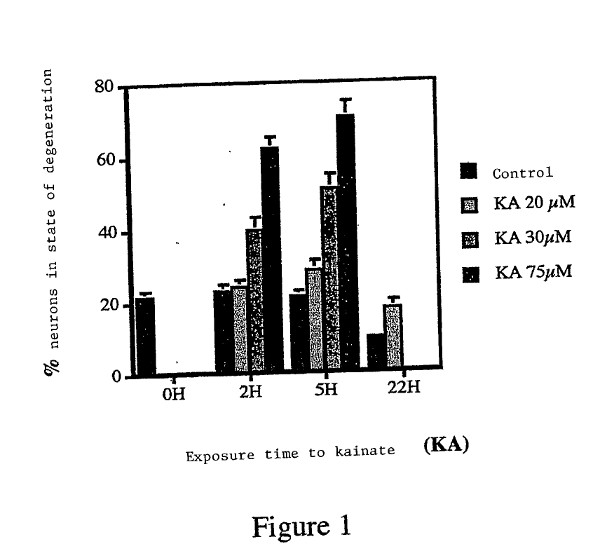

The use of propidium iodine (FIG. 1) at 2, 5 and 22 hours confirmed the morphologic observation data. FIG. 1 represents the kinetic of kainate-dependent neuronal death revealed by propidium iodine. The hippocampus cultures were exposed to different concentrations of kainate (20 μM, 30 μM and 75 μM). The morality peak was at 5 hours after the beginning of kainate treatment. *p<0.05 by ANOVA test.

After exposure to 20 μM of kainate, the percentage of neurons in a state of degeneration increased progressively with a mortality peak at 5 hours. The percentage of neurons in a degenerative state increased in a dose-dependent manner with a maximum mortality at 5 hours after exposure of the cells to kainate at concentrations of 30 to 75 μM. Only some neurons were propidium positive, whereas all of the astrocytes were always propidium negative.

The Hoechst labeling confirmed the data obtained with the propidium iodine.

2) The Protein Cyclin D1 was Expressed in the Vulnerable Neurons After Treatment with Kainate

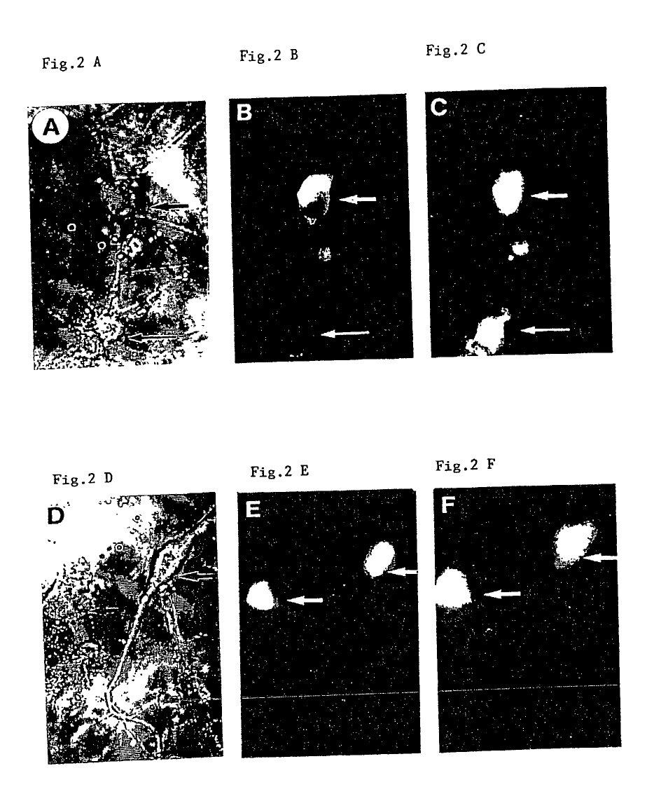

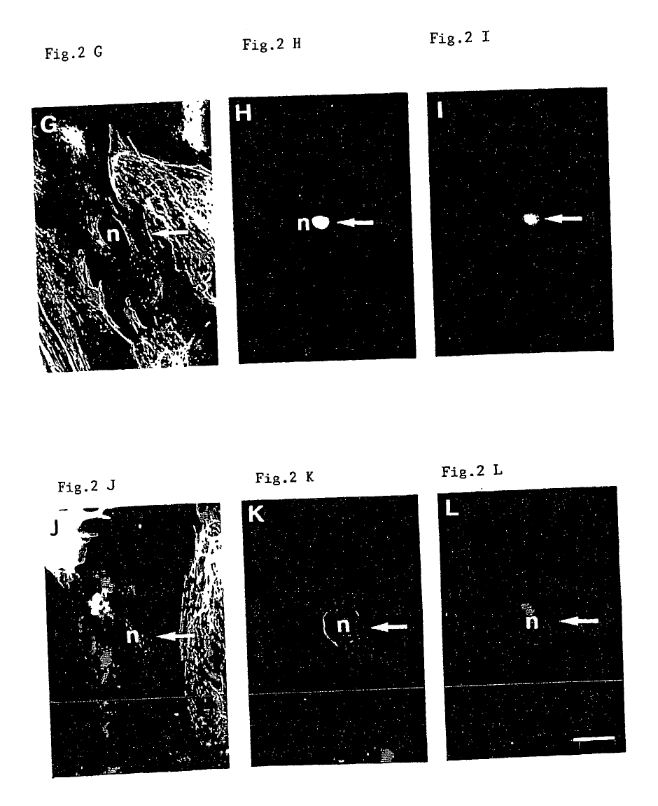

FIG. 2 shows that cyclin D1 was expressed in the vulnerable neurons. Phase-contrast microscope observation and double and triple fluorescent labeling at 22 hours (A-F) and 5 hours (G, H, I) after exposure to 20 μM of kainate, culture not exposed to kainate (J, K, L). Phase-contrast microscope observation (A, D): propidium iodine (B) and cyclin D1 (C, F, I, L); Hoechst labeling (E, H, K); GFAP labeling (G, J). In A, B, C: the neurons (A, arrows) are propidium iodine positive (B) and cyclin D1 positive (C). In D, E, F: the neurons (D, arrows) are Hoechst positive (E) and cyclin D1 positive (F). In G, H, I: one neuron is GFAP positive (I). In J, K, L, an astrocytes is GFAP positive (J), with an uncondensed nucleus (K) and cyclin D1 positive (L). Scale: 1 cm=3.33 μM.

The combination of immunofluorescence observations of cyclin D1 (FIGS. 2C, F) and phase-contrast microscope observation (FIGS. 2A, D) revealed that cyclin D1 was expressed in the neurons. The double labeling of cyclin D1 (FIGS. 2C, F) on the one hand and with propidium iodine (FIG. 2B) or Hoechst dye (FIGS. 2E, H) on the other, revealed that most of the neurons expressing the nuclear protein cyclin D1 presented signs of death revealed by propidium iodine or Hoechst dye (FIGS. 2A-F). Only a few cyclin D1 positive neurons were detected in the control experiments. Moreover, some astrocytes expressed cyclin D1. But the astrocytes (GFAP+) never presented positive propidium iodine labeling or chromatin fragmentation (Hoechst). The control experiments without first antibodies did not reveal any labeling.

3) Expression of the Protein Cyclin D1 was Augmented After Treatment with Kainate

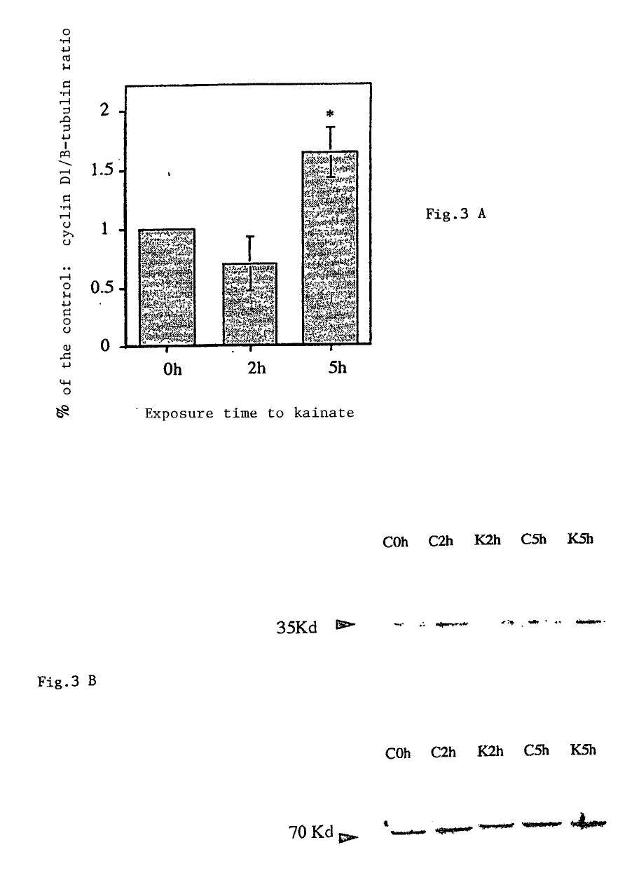

Western blot experiments were performed on cell protein extracts exposed or not exposed to kainate. FIG. 3 shows the augmentation in the normalized level of expression of the protein cyclin D1 after treatment with kainate. FIG. 3 represents the Western blot analyses obtained from protein extracts from hippocampus cells exposed to kainate using the anti-cyclin D1 monoclonal antibody and the class III anti-β-tubulin antibody. In A, abscissa: exposure time (h, hours) to kainate (75 μM). Ordinate: mean level of expression of cyclin D1, normalized by the quantity of neurons, expressed as percentage of the control. The augmentation in the expression of the protein cyclin D1 after 5 hours of exposure to kainate should be noted. *p<0.005 by ANOVA test. In B, representative blots. A band of 35 Kd and a band of 70 Kd were the only bands that could be detected with the anti-cyclin D1 antibody and the class III anti-p-tubulin antibody, respectively.

Since the treatment of the hippocampus cultures with kainate caused neuronal death and thus a loss of neurons, the cyclin D1 level was normalized by the level of class III β-tubulin, a specific marker of the neurons. Quantitative analysis revealed that the normalized level of expression of cyclin D1 increased in a significant manner from 100% before kainate to more than 150% after exposure of the cultures to kainate 75 μM.

4) Cyclin D1 and CDK5 are Co-Expressed in the Neurons in a State of Degeneration and Interact in the Brain

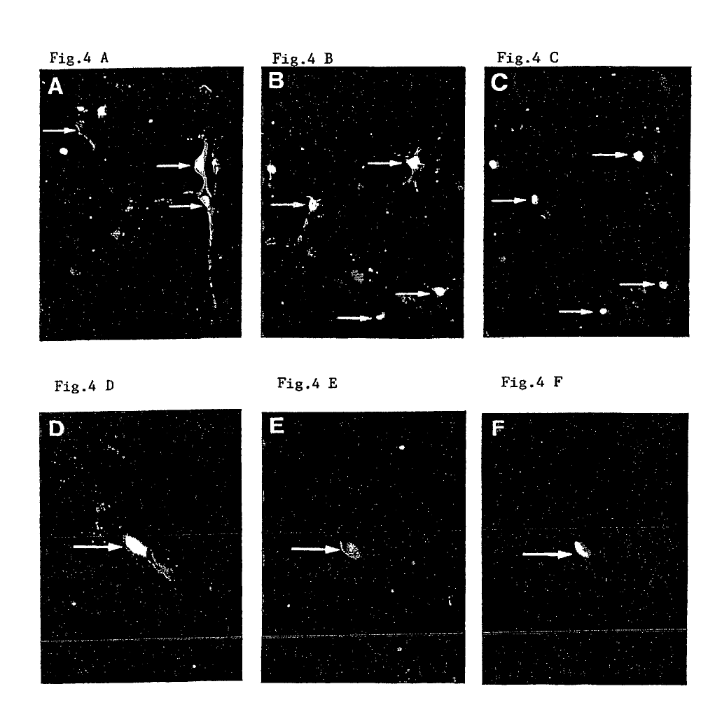

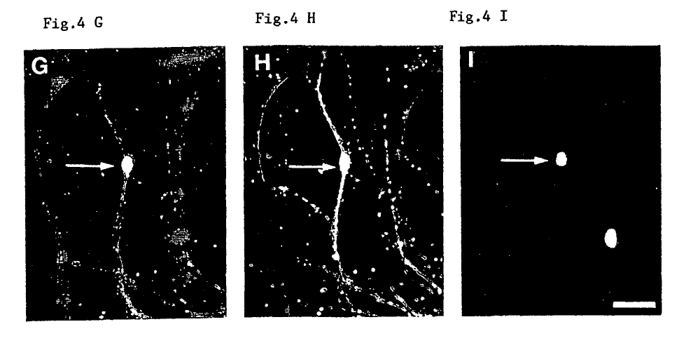

CDK5 is a specifically neuronal kinase dependent cyclin. The cyclin D1/CDK5 double labeling revealed that cyclin D1 and CDK5 were present in the neurons in a state of degeneration. FIG. 4 shows the expression of CDK5 in the neurons after exposure to kainate. Double or triple labeling of hippocampus neurons before (control in A) and after exposure to 75 mM of kainate (B-I). CDK5 immunoreactivity (A, B, D, G); Hoechst dye (C, F, I); propidium iodine (E); cyclin D1 immunoreactivity (H). In A, the neurons (arrows) are CDK5 positive. In B, C, the neurons (arrows) are CDK5 positive (B) with a condensed nucleus (C). In D, E, F, a neuron (arrow), CDK5 positive (D), propidium iodine positive (E) with a condensed nucleus (F). In G, H, I, a neuron (arrow), CDK5 positive (G), cyclin D1 positive (H) with a condensed nucleus (I).

The Western blot studies after immunoprecipitation of CDK revealed that cyclin D1 was associated with CDK5.

5) Effect of CDK Inhibitors on Neuronal Death After Exposure to Kainate

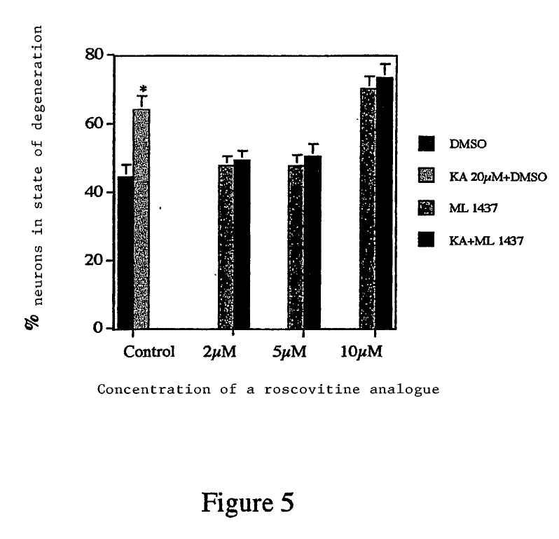

In order to study the role of the cyclin D1/CDK5 complex in neuronal death, a CDK inhibitor that is very active on CDK5 was used on hippocampus cultures exposed to 20 μM of kainate. FIG. 5 shows that a CDK inhibitor decreased the neuronal death after exposure to kainate. FIG. 5 pertains to the hippocampus culture treated for 5 hours by kainate and a CDK inhibitor at different concentrations (2, 5 and 10 μM). Neuronal mortality was evaluated by labeling with propidium iodine with fluorescence microscope observation. It should be noted that neuronal death was partially inhibited by the CDK inhibitor at the concentrations of 2 and 5 μM. *p<0.005 by ANOVA test.

Controls were performed on cultures with or without kainate in combination or not with the CDK inhibitor. In the control experiments with kainate, in the absence of inhibitor, neuronal death was close to 65% with an augmentation of neuronal death by 150% in relation to the cultures without kainate. In contrast, in the cultures with kainate in the presence of CDK inhibitor at a concentration of 2 or 5 μM, neuronal death was close to 45%. The level of neuronal death remained high even with high doses of inhibitors (10 μM).

III—Discussion

The initial studies (Timsit et al., 1999) showed that the expression of cyclin D1 was augmented in vivo in vulnerable neurons but also, at a lower level, in resistant neurons. Thus, it could not be demonstrated whether this expression had a deleterious or beneficial effect. The in vitro studies of this invention have confirmed augmentation of the expression of the protein cyclin D1 after exposure of cultures of neurons and astrocytes to kainate, an analogue of glutamate. Moreover, the immunohistochemistry studies demonstrated that the neurons in the state of degeneration express the protein cyclin D1 in their nuclei. This expression occurs at an early stage with the fragmentation of the DNA as had already been shown in the in vivo studies. The cyclin D1/CDK5 double labeling study demonstrated that the neurons in the state of degeneration co-express these two proteins, suggesting that that they could be associated. The Western blot study on normal rat brains confirmed the possibility of an association between cyclin D1 and the CDK5 molecule. Finally, use of a CDK inhibitor that is preferentially active on CDK5 showed a protective effect of this chemical product at doses between 2 and 5 μM. In contrast, at the dose of 10 μM this product was no longer found to have a protective effect.

The morphologic appearances associated with kainate analyzed with phase-contrast microscopy, with a Hoechst marker and propidium iodine, demonstrated both the aspects of apoptosis and the aspects of necrosis. The apoptosis aspects are characterized by the condensation and fragmentation of the nucleus visualized by Hoechst coloration, but also necrosis aspects with rupture of the cytoplasmic membrane visualized by propidium iodine coloration. Thus, the CDK inhibitors have a neuroprotective effect against neuronal excitotoxicity that is not typically apoptotic. These data are furthermore supported by the work of Leski et al. (1999) which showed that the excitotoxic neuronal death induced by kainate can not be prevented by the use inhibitors of the synthesis of RNA or protein or inhibitors of caspases such as YVAD-CHO and DEVD-CHO. Thus, the classic criteria generally associated with apoptosis, i.e., programmed death and activation of caspases, are not found in the excitotoxic death induced by kainate. Moreover, the inhibitors of caspases are not always active on the models of cerebral ischemia. Thus, Li et al. (2000) demonstrated an absence of effect of caspase inhibitors in global ischemia.

### BIBLIOGRAPHIC REFERENCES

The subject matter of the publications listed below is hereby incorporated by reference.

Khrestchatisky, M., Timsit, S., Rivera, S., Tremblay, E. and Ben-Ari, Y. (1996), Neuronal death and damage repair: roles of protoncogenes and cell cycle-related proteins. In J. Krieglstein (Ed.), Pharmacology of Cerebral ischemia. Medpharm Scientific Publishers, Stuttgart, pp 41-56.

Li, Y., Chopp, M., Powers, C., Jiang, N. (1997), Immunoreactivity of cyclin D1/cdk4 in neurons and oligodendrocytes after focal cerebral ischemia in rats. J Cereb Blood Flow Metab., 17: 846-856.

Sherr, C. J. (1993), Mamalian G1 cyclins. Cell, 73: 1059-1065.

Zwijsen, R. M., Wientjens, E., Klompmaker, Van der Sman J., Bernards, R., Michalides, R. J. A. M. (1997), Cdk-independant activation of estrogen receptor by cyclin D1. Cell, 88: 405-415.

Portera-Cailliau, Price D. L., Martin, L. J., Non-NMDA and NMDA receptor mediated excotoxic neuronal deaths in adult brain are morphologically distinct: further evidence for an apoptotic-necrosis continuum, 1997. J. Comp. Neurol 378: 88-104.

Wiessner, C., Brink, I., Lorenz, P., Neumann-Haefelin, T., Vogel, P., Yamashita, K. (1996), Cyclin D1 messenger RNA is induced in microglia rather than neurons following transient forebrain ischemia. Neuroscience, 72: 947-958.

Small, D. L., Monette, R., Comas, T., Fournier, M. C., Morley, P. (1999), Loss of cyclin D1 in necrotic and apototic models of cortical neuronal degeneration. Brain Research, 842: 376-383.

Timsit, S., Rivera, S., Ouaghi, P., Guischard, F., Tremblay, E., Ben-Ari, Y., Khrestchatisky, M., Increased Cyclin D1 in vulnerable neurons in the hippocampus after ischemia and epilepsy: a modulator of in vivo programmed cell death, (1999). Eur. J. Neurosci., 1999, 11, 263-278.

Oda, H., Kasiske, B. L., O'Donnell, M. P., Keane, W. F. (1999), Effects of lovastatine on expression of cell cyle regulatory proteins in vascular smooth muscle cells. Kindney Int Suppl 71, S202-S205.

Rao, S., Porter, D. C., Chen, X., Herliczek, T., Lowe, M., Keyomarsi, K. (1999), Effects of lovastatin on expression of cell cycle regulatory proteins in vascular smooth musle cells. Proc. Natl. Acad. Sci., 96, 7797-7802.

Muller, C., Kiehl, M. G., van de Loo, J., Koch, O. M. (1999), Lovastatin induces p21WAF1/Cip1 in human vascular smooth muscle cells: influence on protein phophorylation, cell cycle, induction of apoptois and growth inhibition.

Hashemolhosseini, S., Nagamine, Y., Morley, S. J., Desriviires, S., Mercep, L., Ferrari, S. (1998), Rapamycin inhibition of the G1 to S transition is mediated by effects on cyclin D1 mRNA and protein stability. J. Biol. Chem. 273, 14424-14429.

Diehl, J. A., Cheng, M., Roussel, M. F., Sherr, C. J., Glycogen synthase kinase-3beta regulates cyclin D1 prteolysis and subcellular localization, (1998). Genes Dev. 22, 3499-3511.

Borner, C. and Monney, L., Apoptosis without caspases: an efficient molecular guillotine. Cell death Differ 6, 508-515 (1999).

Kitanaka, C., Kuchino, Y., Caspases independant programmed cell death with necrotic morphology. Cell death Differ, 6, 508-515 (1999).

Chautan, M., Chazal, G., Ceconni, F., Gruss, P., Golstein, P., Interdigital cell death occur through a necrotic and caspase-independant pathway, (1999). Curr Biol 9, 967-970.

MacManus, J. P., Fliss, H., Preston, E., Rasquinha, I., Tuor, U. (1999), Cerebral ischemia produces laddered DNA fragment distinct from cardiac ischemia and archetypal apoptosis. J. Cereb. Blood Flow Metab. 19: 502-510.

Leski, M. L., Valentine, S. L., Coyle, J. T. (1999), L-type voltage gated calcium channels modulate kainic acid neurotoxicity in cerebellar granule cells. Brain Res. 828: 27-40.

Li, H., Colbourne, F., Sun, P., Zhao, Z., Buchan, A. M., Iadecolar, C. (2000), Caspase inhibitors reduce neuronal injury after focal but not global cerebral ischemia in rats. Stroke 31: 176-82.

del Zoppo, G., Ginis, I., Hallenbeck, J. M., Iadecola, C., Wan, X., Feuerstein, G. Z., Inflammation and stroke: putative role for cytokines, adhestion molecules and iNOS in brain response to ischemia, (2000). Brain pathology 10: 95-112.

Sanchez-Gomez, M. V., Matute, C., AMPA and kainate receptors each mediate excitotoxicity in oligodendroglial cultures, (1999). Neurobiol Dis. 6: 475-485.

Matute, C., Sanchez-Gomez, M. V., Martinex-Millan, L., Mideldi, R. (1997), Glutamate receptor-mediated toxicity in optic nerve oligodendrocytes. P.N.A.S., 94: 8830-8835.

Kerr, J. F. R., Willie, A. H., Currie, A. R. (1972), Apoptosis: a basic biological phenomenon with wide ranging implications in tissue kinetic. Br. J. Cancer 26, 239-257.

1. A method of treating or preventing non-apoptotic excitotoxic acute neural lesions in a patient comprising administering a therapeutically effective amount of a substance that modulates expression or the function of a protein involved in cell cycles.

2. A method of treating or preventing non-apoptotic excitotoxic acute neural lesions of neurons, astrocytes or oligodendrocytes or their precursors over the course of epilepsy comprising administering a therapeutically effective amount of a substance that modulates expression or the function of a protein involved in cell cycles.

3. A method of treating or preventing non-apoptotic excitotoxic acute neural lesions of neurons, astrocytes or oligodendrocytes or their precursors during cerebral ischemia comprising administering a therapeutically effective amount of a substance that modulates expression or the function of a protein involved in cell cycles.

4. The method according to claim 3, wherein the cerebral ischemia occurs from cerebral hypoxia or anoxia.

5. The method according to claim 4, wherein the cerebral hypoxia or anoxia is caused by an event selected from the group consisting of cardiac arrest, implementation of extracorporeal circulation during cardiovascular surgery, surgery of the vessels of the neck possibly requiring clamping of vessels and cranial trauma.

6. The method according to claim 1, wherein the protein involved in the cell cycle is a protein required for progression of the cell cycle.

7. The method according to claim 1, wherein the protein involved in the cell cycle is produced by a cell that is capable or incapable of dividing.

8. The method according to claim 1, wherein the substance is capable of modulating phosphorylation of a target by augmenting or inhibiting the phosphorylation.

9. The method according to claim 1, wherein the substance modulates expression or the function of a cyclin and/or a CDK.

10. The method according to claim 1, wherein the substance modulates expression or the function of a D cyclin and/or a CDK.

11. The method according to claim 1, wherein the substance modulates expression or the function of cyclin D1 and/or CDK5 and/or a cyclin D1/CDK5 complex.

12. The method according to claim 1, wherein the substance is selected from the group consisting of:
inhibitors of expression of cyclins,
inhibitors of cyclin dependent kinases,
inhibitors of a cyclin/cyclin dependent kinase complex.

13. The method according to claim 12, wherein the inhibitor of the expression of cyclins is selected from the group consisting of rapamycin, glycogen synthase kinase and statins.

14. The method according to claim 12, wherein the inhibitor of cyclin dependent kinases is selected from the group consisting of analogues of purines, paullones, indirubins, hymenisaldisine and flavopiridol.

15. A pharmaceutical preparation for treating or preventing non-apoptotic excitotoxic acute neural lesions comprising a therapeutically effective amount of a substance that modulates expression or the function of a protein involved in the cell cycle.

16. A pharmaceutical preparation for treating or preventing non-apoptotic excitotoxic acute neural lesions of neurons, astrocytes or oligodendrocytes or their precursors over the course of epilepsy comprising a therapeutically effective amount of a substance that modulates the expression or the function of a protein involved in the cell cycle.

17. A pharmaceutical preparation for treating or preventing non-apoptotic excitotoxic acute neural lesions of neurons, astrocytes or oligodendrocytes or their precursors during cerebral ischemia comprising a theapeutically effective amount of a substance that modulates the expression or the function of a protein involved in the cell cycle.

18. The pharmaceutical preparation according to claim 15, wherein the protein is a protein required for the progression of the cell cycle.

19. The pharmaceutical preparation according to claim 15, wherein the protein is produced by a cell that is capable or incapable of dividing.

20. The pharmaceutical preparation according to claim 15, wherein the substance is capable of modulating phosphorylation of a target by augmenting or inhibiting the phosphorylation.

21. The pharmaceutical preparation according to claim 15, wherein the substance modulates the expression or the function of a cyclin and/or a CDK.

22. The pharmaceutical preparation according to claim 15, wherein the substance modulates the expression or the function of a D cyclin and/or a CDK.

23. The pharmaceutical preparation according to claim 15, wherein the substance modulates the expression or the function of cyclin D1 and/or CDK5 and/or a cyclin D1/CDK5 complex.

24. The pharmaceutical preparation according to claim 15, wherein the substance is selected from the group consisting of:
inhibitors of expression of cyclins,
inhibitors of cyclin dependent kinases,
inhibitors of a cyclin/cyclin dependent kinase complex.

25. The pharmaceutical preparation according to claim 24, wherein the inhibitor of the expression of cyclins is selected from the group consisting of rapamycin, glycogen synthase kinase and stations.

26. The pharmaceutical preparation according to claim 24, wherein the inhibitor of cyclin dependent kinases is selected from the group consisting of analogues of purines, paullones, indirubins, hymenisaldisine and flavopiridol.
